(19) United States
(12) Patent Application Publication
(10) Pub. No.: US 2011/0200645 A1
(43) Pub. Date: Aug. 18, 2011

(54) DHA Free Fatty Acid Emulsions

(75) Inventors: Jung LEE, McLean, VA (US); Bernard A. Mikrut, Lake Bluff, IL (US); Xuejun Tang, Ellicott City, MD (US)

(73) Assignee: Martek Biosciences Corporation, Columbia, MD (US)

(21) Appl. No.: 13/031,178

(22) Filed: Feb. 18, 2011

Related U.S. Application Data
(60) Provisional application No. 61/305,951, filed on Feb. 18, 2010.
(60) Provisional application No. 61/361,311, filed on Jul. 2, 2010.
(60) Provisional application No. 61/367,350, filed on Jul. 23, 2010.

(51) Int. Cl. A61K 9/107; A61K 31/202; A61P 9/10; A61P 19/02; A61P 1/16; A61P 9/04

(52) U.S. Cl. 424/400; 514/560

(57) ABSTRACT

The present invention is directed to an emulsion comprising an emulsifier, an isotonic agent and a docosahexaenoic acid free fatty acid (DHA-FFA) wherein the emulsion is substantially free of eicosapentaenoic acid (EPA) and is suitable for parenteral administration.

This application claims the benefit of the filing date of U.S. Appl. No. 61/305,951, filed Feb. 18, 2010, U.S. Appl. No. 61/361,311, filed Jul. 2, 2010, and U.S. Appl. No. 61/367,350, filed Jul. 23, 2010, all of which are incorporated by reference.

### BACKGROUND

### Field of the Invention

The present invention is directed to emulsions comprising docosahexaenoic acid free fatty acid (DHA-FFA) for parenteral administration.

### BRIEF SUMMARY

An emulsion comprising an emulsifier, an isotonic agent and a docosahexaenoic acid free fatty acid (DHA-FFA) wherein the emulsion is substantially free of eicosapentaenoic acid (EPA) and is suitable for parenteral administration. In some embodiments the emulsion comprises a secondary emulsifier.

Also provided herein is a method of making an emulsion comprising dispersing an emulsifier and an isotonic agent in water to form a coarse dispersion; homogenizing the coarse dispersion to form a fine dispersion; mixing oil containing DHA-FFA to the dispersion, more particularly to the fine dispersions, to form a coarse emulsion. Homogenizing the coarse emulsion to form the emulsion. In some embodiments the pH is adjusted to about 6 to about 9. The final emulsion can be autoclaved. In some embodiments a secondary emulsifier is mixed with the emulsion, more particularly to the coarse emulsion.

### DETAILED DESCRIPTION

For the descriptions herein and the appended claims, the singular forms “a”, “an” and “the” include plural referents unless the context clearly indicates otherwise. Thus, for example, reference to “a compound” refers to more than one compound.

Also, the use of “or” means “and/or” unless stated otherwise. Similarly, “comprise,” “comprises,” “comprising,” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Provided herein is an emulsion comprising an emulsifier, an isotonic agent and a docosahexaenoic acid free fatty acid (DHA-FFA) wherein the emulsion is substantially free of eicosapentaenoic acid (EPA) and is suitable for parenteral administration. In some embodiments the emulsion further comprises a secondary emulsifier.

In some embodiments provided herein the concentration of the DHA-FFA is about 0.2 milligram per milliliter (mg/ml) to about 300 mg/ml of the emulsion. In some embodiments the concentration of the DHA-FFA is about 0.3 mg/ml to about 300 mg/ml of the emulsion. In some embodiments the concentration of the DHA-FFA is about 20 milligram per milliliter (mg/ml) to about 300 mg/ml of the emulsion. In some embodiments the concentration of the DHA-FFA is about 30 mg/ml to about 300 mg/ml. In some embodiments, the concentration of the DHA-FFA is about 0.2 mg/ml to about 30 mg/ml of the emulsion. In some embodiments, the concentration of DHA-FFA is about 140 to about 300 mg/ml of the emulsion. In some embodiments, the concentration of DHA-FFA is about 140 to about 150 mg/ml of the emulsion. In some particular embodiments, the concentration of the DHA-FFA is about 140 mg/ml to about 143 mg/ml of the emulsion. In some embodiments, the concentration of the DHA-FFA is about 270 mg/ml to about 300 mg/ml of the emulsion. In some embodiments, the concentration of the DHA-FFA is about 280 mg/ml to about 300 mg/ml of the emulsion. In some particular embodiments, the concentration of DHA-FFA is about 280 to about 290 mg/ml of the emulsion.

In some embodiments, the emulsion comprises about 2% to about 30%, by weight, oil containing the DHA-FFA. In some embodiments the emulsion comprises about 0.3% to about 30%, by weight, oil containing the DHA-FFA. In some embodiments, the emulsion comprises about 3% oil to about 30%, by weight, oil containing the DHA-FFA. In some embodiments the emulsion comprises about 3% by weight oil containing the DHA-FFA. In some embodiments the emulsion comprises about 15% to about 30% by weight of the oil containing the DHA-FFA. In some embodiments the oil in the emulsion comprises, by weight, about 84% to about 99% DHA-FFA. In some particular embodiments the emulsion comprises about >95% DHA-FFA of the total weight of the oil.

In some embodiments the concentration of the DHA-FFA is about 0.3 mg/ml of the emulsion.

In some embodiments, the concentration of the DHA-FFA is about 3.0 mg/ml of the emulsion.

In some embodiment, the concentration of the DHA-FFA is about 30 mg/ml of the emulsion.

In some embodiments, the above oil and/or DHA-FFA concentrations can be obtained by starting with a low concentration of oil (e.g., but not limited to 3% by weight) or higher (e.g., but not limited to 30% weight) and then diluted for example into water, the dilution factor including, for example but noted limited to, a thousand fold, a hundred fold, and more particularly ten fold.

In some embodiments provided herein, the mean particle size of the emulsion is about 500 nanometers. In some embodiments, the emulsions provided herein have a mean diameter size of less than about 500 nanometers (or 0.5 μm). In some embodiments, the emulsions provided herein have a percentage of fat residing in globules larger than 5 μm (PFAT5) is 0.05% or less. Examples of globule size distribution limits and their determination (e.g., mean diameter and large-diameter tail) of an injectable emulsion useful for total parenteral nutrition can be found for example in Chapter 729 of the United States Pharmacopeia (USP).

In particular embodiments, the mean particle size is about 100 to about 200 nanometers.

In some embodiments the change in uniformity measurement of the emulsion is less than or equal to about 10%, more particularly 5% after two months at room temperature.

In some embodiments, the change in mean diameter of the emulsion is less than or equal to about 10%, more particularly 5% after two months at room temperature.

In some embodiments, the PFAT5 of the emulsion is about 0.05% or less after two months at room temperature.

In some embodiments provided herein, the emulsion comprises about 0.6% to about 10%, by weight, of the emulsifier. In some embodiments, the emulsion comprises about 1 to about 4%, by weight, of the emulsifier. Particularly, in some embodiments the emulsion comprises about 1.8 or about 3.6%, by weight, of the emulsifier. Emulsifiers that are suitable for parenteral use (e.g., physiologically safe) can be used in embodiments provided herein. Non-limiting examples of emulsifiers include phospholipids of animal or vegetable origin. Other non-limiting examples include a lecithin including, but not limited to, synthetic and semi-synthetic lecithins. Egg phospholipid mixtures, such as Lipoid E-80 SN (Lipoid GmbH, Ludwigshafen, Germany), are particular examples of an emulsifier provided herein.

An isotonic agent can be added to adjust the osmolarity of the emulsion to a desired physiologically acceptable level. In some embodiments, the emulsion has an osmolarity of about 280 to about 300 milliosmols/liter, particularly about 300 milliosmol/liter. In some embodiments, the emulsion comprises about 1% to about 5%, by weight, of the isotonic agent. In some embodiments, the emulsion comprises about 1% to about 2.5%, by weight, of the isotonic agent. Particularly, in some embodiments the emulsion comprises about 2.25% to about 2.5%, by weight, of the isotonic agent. Examples of suitable isotonic agents include, but are not limited to, glycerin, glucose, xylose, and sorbitol. In some embodiments, the particular isotonic agent comprises glycerin.

In some embodiments the secondary emulsifier comprises about 0.03% to about 0.4%, by weight, more particularly about 0.03% to about 0.3%, by weight, of the emulsion. In some embodiments suitable secondary emulsifiers that can be used for example are linoleic acid, linolenic acid, oleic acid, palmitic acid or their pharmaceutically acceptable salts (e.g., but not limited to potassium and sodium). In some embodiments the secondary emulsifier is sodium oleate. In some embodiments the sodium oleate is provided in an amount of about 0.3% (equivalent to about 3 mg/ml) by weight.

In some embodiments, an oil comprising triglycerides is added to the emulsion in an amount sufficient to provide a PFAT5 value for the emulsion of about 0.05% or less. In some embodiments, the oil containing a triglycerides is provided in an amount greater than about 0.5%, more particularly from about 0.5% to 3.3%, and more particularly about 3.3% by weight of the emulsion. In some embodiments, the triglycerides content of the oil is greater than 90% by weight.

In some embodiments, the emulsion comprises about 150 milligrams of DHA-FFA per milliliter of the emulsion, about 18 milligrams of a lecithin per milliliter of the emulsion; and about 25 milligrams of glycerin per milliliter of the emulsion wherein the emulsion has a mean particle size up to and including about 500 nanometers, more particularly about 100 to about 200 nanometers, wherein the emulsion is substantially free of EPA and is suitable for parenteral administration.

In some embodiments, the emulsion comprises about 300 milligrams of DHA-FFA per milliliter of the emulsion, about 36 milligrams of a lecithin per milliliter of the emulsion; and about 50 milligrams of glycerin per milliliter of the emulsion wherein the emulsion has a mean particle size up to and including about 500 nanometers, more particularly about 100 to about 200 nanometers, wherein the emulsion is substantially free of EPA and is suitable for parenteral administration.

In some embodiments, the emulsion can also include antioxidants and other agents, including but not limited to Vitamin E, Vitamin C, carotenoids, flavonoids, lipoic acid, tocotrienols, and tocopherols. Other physiologically safe additives can also be used in some embodiments including, but not limited to, common intravenous salts such as sodium chloride and nonelectrolytes such as glucose, pH modifiers (such as acetic acid and sodium acetate) and buffers (such as acetate, lactate, and phosphate buffer systems composed of the acid and a salt of the acid), emulsion stabilizers like gelatin, polysaccharides, such as agar, and/or detergents like tweens and spans, as well as selenium compounds. In some embodiments, the emulsion is provided substantially free of detergents, for example non-ionic detergents, e.g., tweens.

In some embodiments the emulsion is made by mixing an oil containing a DHA free fatty acid, an isotonic agent, an emulsifier and water and further homogenizing the mixture to a desired particle size. The pH of the emulsion can be adjusted for example to a desired pH. For example, in some embodiments, the emulsion has a pH of about 5 to about 9, particularly about 7 to about 9. In some embodiments, the emulsion has a pH of 6.5 to about 8.5, more particularly about 7 to about 8. In some embodiments, the pH is adjusted with a pH adjuster that is suitable for parenteral use, for example, but not limited to sodium hydroxide.

In some embodiments, an emulsion is provided substantially free of a therapeutic amount of an active agent other than DHA-FFA. In some embodiments, an emulsion is provided in the absence of a therapeutic amount of an anti-cancer agent.

In some embodiments, an emulsion is provided substantially free of a medium chain fatty acid, in particular a medium chain triglyceride. In some embodiments, the medium chain fatty acid is present in an amount less than about 10% (w/w), less than about 5% (w/w), less than about 2% (w/w), or less than about 1% (w/w) of the total fatty acid content of the emulsion. In some embodiments there is no detectable medium chain fatty acid, in particular no detectable medium chain triglyceride.

In some embodiments, chelating agents, such as ethylenediaminetetraacetic acid (EDTA) and its derivatives including, but not limited to their pharmaceutically acceptable salts, are present in the emulsion. Derivatives are meant to encompass structural analogs, for example, but not limited to, diethylenetriaminepentaacetic acid (DTPA) and its pharmaceutically acceptable salts.

In some embodiments, preservatives, such as benzyl alcohol or sodium benzoate, are present in the emulsion.

Some embodiments provided herein can be used for therapeutic purposes.

In some embodiments, the emulsions provided herein can be provided in an effective amount to treat a subject suffering from traumatic brain injury, including but limited to, a closed head injury, such as a concussion or a contusion; or a penetrating head injury. The type of traumatic head injury can be mild, moderate or severe, and involve diffuse axonal injury or hematoma.

Some embodiments of the emulsions provided herein are useful to treat subjects suffering from spinal cord injury.

Some embodiments provided herein can be used to treat a subject suffering from ischemic brain injury including but not limited to stroke. Some embodiments can be used to treat a subject suffering from a hemorrhagic stroke or other types of brain trauma associated with bleeding.

In some embodiments, the emulsions provided herein can be used to treat inflammatory conditions including, but not limited to arthritis. Arthritis is defined herein as inflammatory diseases of the joints, including, but not limited to osteoarthritis, gouty arthritis, ankylosing spondylitis, psoriatic arthritis, reactive arthritis, rheumatoid arthritis, juvenile onset rheumatoid arthritis, infectious arthritis, inflammatory arthritis, septic arthritis, degenerative arthritis, arthritis mutilans, and lyme arthritis.

In some embodiments, the emulsions provided herein can be used to treat a subject suffering from liver disorders such as fatty liver (hepatosteatosis). In some embodiments the liver disorder includes, but is not limited to, nonalcoholic fatty liver disease (NAFLD). NAFLD refers liver diseases including, but not limited to, simple fatty liver (hepatosteatosis), nonalcoholic steatohepatitis (NASH), and cirrhosis (irreversible, advanced scarring of the liver), that result from accumulation of fat in liver cells, that is not due to excessive alcohol intake. Hepatosteatosis is the accumulation of fat in the liver. Steatohepatitis is characterized by fat accumulation in the liver concurrent with hepatic inflammation. In some embodiments, the emulsions provided herein can be used to treat a subject suffering from steatohepatitis, resulting from excessive alcohol intake. In some embodiments, an emulsion provided here can be used to treat a subject suffering from primary sclerosing cholangitis.

In some embodiments, the subject has e.g., hepatosteatosis, hepatic inflammation, cirrhosis, biliary obstruction, and/or hepatic fibrosis. In some embodiments, it is desirable to treat, e.g., to reduce hepatosteatosis, hepatic inflammation, cirrhosis, biliary obstruction, and/or hepatic fibrosis; prevent hepatosteatosis, hepatic inflammation, cirrhosis, biliary obstruction, and/or hepatic fibrosis; or retard the onset of hepatosteatosis, hepatic inflammation, cirrhosis, biliary obstruction, and/or hepatic fibrosis.

In some embodiments, the emulsions provided herein can be used to treat hepatic fibrosis. In some embodiments, the emulsions provided herein can be used to prevent formation of new fibroids. In some embodiments, the emulsions provided herein can be used to can be used to reduce the number of fibroids. In some embodiments, the emulsions provided herein can be used to retard the onset of fibroid formation.

In some embodiments, the emulsions provided herein can be used to treat a subject suffering from congestive heart failure, including both chronic and acute congestive heart failure. In some embodiments, the emulsions provided herein can be used to treat heart arrhythmia originating in either the atrium or the ventricle.

In some embodiments, the emulsions provided herein can be used to prevent or reduce the risk of post-operative cognitive dysfunction in a subject.

Provided herein are emulsions for parenteral use. “Suitable for parenteral administration” refers to compositions, e.g., emulsions, that are, within the scope of sound medical judgment, suitable for parenteral administration into human beings and/or animals without excessive toxicity or other complications commensurate with a reasonable benefit/risk ratio. In some embodiments, “suitable for parenteral administration” refers to an emulsion which is deemed physiologically safe, or safe for human administration, by a governmental entity, e.g., the United States Food and Drug Administration. An example of a definition of parenteral can be found for example in Stedman's Medical Dictionary, 26^{th} Edition. In some embodiments, parenteral administration of an emulsion provided herein refers particularly to the introduction of the emulsion into a subject by intravenous, subcutaneous, intramuscular, or intramedullary injection. In some embodiments an emulsion provided herein can be administered to a subject as a bolus injection. In some embodiments the bolus injections comprise about 1 ml to about 50 ml of an emulsion provided herein. In some embodiment, an emulsion is administered to a subject by at least one 5 ml bolus dose. In some embodiments the bolus injection can comprise about 5 ml of an emulsion provided herein. In some embodiments, an emulsion can be administered intravenously (IV) to a subject. In some embodiments, the IV administration can be infused continuously. A particular amount of DHA-FFA in an emulsion herein that can be administered parenterally to a subject can range about 0.1 gram to about 20 grams. In some embodiments, administration of the emulsion can be prescribed by a medical professional.

The term “subject” refers to mammals such as humans or primates, such as apes, monkeys, orangutans, baboons, gibbons, and chimpanzees. The term “subject” can also refer to companion animals, e.g., dogs and cats; zoo animals; equids, e.g., horses; food animals, e.g., cows, pigs, and sheep; and disease model animals, e.g., rabbits, mice, and rats. The subject can be a human or non-human. The subject can be of any age. For example, in some embodiments, the subject is a human infant, i.e., post natal to about 1 year old; a human child, i.e., a human between about 1 year old and 12 years old; a pubertal human, i.e., a human between about 12 years old and 18 years old; or an adult human, i.e., a human older than about 18 years old. In some embodiments, the subject is an adult, either male or female.

As used herein, the terms “treat” and “treatment” refer to both therapeutic treatment and prophylactic or preventative measures, wherein the object is to prevent or slow down (lessen) an undesired physiological condition or disease, or obtain beneficial or desired clinical results. The term “treatment” also refers to the alleviation of symptoms associated with the above conditions or diseases.

In some embodiments, the DHA-FFA is administered continuously. The term “continuous” or “consecutive,” as used herein in reference to “administration,” means that the frequency of administration is at least once daily. Note, however, that the frequency of administration can be greater than once daily and still be “continuous” or “consecutive,” e.g., twice or even three or four times daily, as long as the dosage levels as specified herein are achieved.

“DHA” refers to docosahexaenoic acid, also known by its chemical name (all-Z)-4,7,10,13,16,19-docosahexaenoic acid, as well as any salts or derivatives thereof. Thus, the term “DHA” encompasses the free acid DHA (DHA-FFA) as well as DHA phospholipids, esters, monoglycerides, diglycerides, and triglycerides containing DHA. DHA is an ω-3 polyunsaturated fatty acid. The DHA can be in a mono, di, or triglyceride form. For example, one, two or three DHA molecules can be in the mono, di or triglyceride molecule.

The term “ester” herein refers to the replacement of the hydrogen in the carboxylic acid group of the DHA molecule with another substituent. Typical esters are known to those in the art. Examples of the most common esters include methyl, ethyl, propyl, butyl, pentyl, t-butyl, benzyl, nitrobenzyl, methoxybenzyl, benzhydryl, and trichloroethyl. In some embodiments, the ester is a carboxylic acid protective ester group, esters with aralkyl (e.g., benzyl, phenethyl), esters with lower alkenyl (e.g., allyl, 2-butenyl), esters with lower-alkoxy-lower-alkyl (e.g., methoxymethyl, 2-methoxyethyl, 2-ethoxyethyl), esters with lower-alkanoyloxy-lower-alkyl (e.g., acetoxymethyl, pivaloyloxymethyl, 1-pivaloyloxyethyl), esters with lower-alkoxycarbonyl-lower-alkyl (e.g., methoxycarbonylmethyl, isopropoxycarbonylmethyl), esters with carboxy-lower alkyl (e.g., carboxymethyl), esters with lower-alkoxycarbonyloxy-lower-alkyl (e.g., 1-ethoxycarbonyloxy)ethyl, 1-(cyclohexyloxycarbonyloxy)ethyl), esters with carbamoyloxy-lower alkyl (e.g., carbamoyloxymethyl), and the like. In some embodiments, the added substituent is a linear or cyclic hydrocarbon group, e.g., a C₁-C₆ alkyl, C₁-C₆ cycloalkyl, C₁-C₆ alkenyl, or C₁-C₆ aryl ester.

In some embodiments, the ester is an alkyl ester, e.g., a methyl ester, ethyl ester or propyl ester. More particularly, the ester is an ethyl ester. The ester substituent can be added to the DHA free acid molecule when the DHA is in a purified or semi-purified state. Alternatively, the DHA ester is formed upon conversion of a triglyceride to a ester. One of skill in the art can appreciate that some non-esterified DHA molecules can be present in the present invention, e.g., DHA molecules that have not been esterified, or DHA linkages that have been cleaved, e.g., hydrolyzed.

In some embodiments, the oil containing DHA, or emulsion containing DHA-FFA is substantially free of eicosapentaenoic acid (EPA). EPA refers to eicosapentaenoic acid, known by its chemical name (all-Z)-5,8,11,14,17-eicosapentaenoic acid, as well as any salts or derivatives thereof. Thus, the term “EPA” encompasses the free acid EPA as well as EPA alkyl esters and triglycerides containing EPA. EPA is an ω-3 polyunsaturated fatty acid. As used herein, an oil “substantially free of EPA” can refer to an oil in which EPA is less than about 3%, by weight, of the total fatty acid content of the oil. In some embodiments, the oil comprises less than about 2% EPA, by weight, of the total fatty acid content of the oil, less than about 1% EPA, by weight, of the total fatty acid content of the oil, less than about 0.5% EPA, by weight, of the total fatty acid content of the oil, less than about 0.2% EPA, by weight, of the total fatty acid content of the oil, or less than about 0.01% EPA, by weight, of the total fatty acid content of the oil. In some embodiments, the oil has no detectable amount of EPA. As used herein, a emulsion “substantially free of EPA” can refer to an emulsion in which EPA is less than about 3%, by weight, of the total fatty acid content of the emulsion. In some embodiments, the emulsion comprises less than about 2% EPA, by weight, of the total fatty acid content of the emulsion, less than about 1% EPA, by weight, of the total fatty acid content of the emulsion, less than about 0.5% EPA, by weight, of the total fatty acid content of the emulsion, less than about 0.2% EPA, by weight, of the total fatty acid content of the emulsion, or less than about 0.01% EPA, by weight, of the total fatty acid content of the emulsion. In some embodiments, the emulsion has no detectable amount of EPA.

With respect to comparison of DHA to total fatty acid content, weight % can be determined by calculating the area under the curve (AUC) using standard means, e.g., dividing the DHA AUC by the total fatty acid AUC.

In some embodiments, the oil containing DHA, or emulsion containing DHA-FFA, is substantially free of docosapentaenoic acid 22:5n-6 (DPAn6). The term DPAn6 refers to docosapentaenoic acid, omega 6, known by its chemical name (all-Z)-4,7,10,13,16-docosapentaenoic acid, as well as any salts or esters thereof. Thus, the term DPAn6 encompasses the free acid DPAn6, as well as DPAn6 ethyl esters and triglycerides containing DPAn6. DPAn6 can be removed during purification of DHA, or alternatively, the DHA can be obtained from an organism that does not produce DPAn6, or produces very little DPAn6. As used herein, an oil “substantially free of DPAn6” refers to an oil containing less than about 2%, by weight, docosapentaenoic acid 22:5n-6 (DPAn6) of the total fatty acid content of the oil. In some embodiments, the oil contains less than about 1% DPAn6, by weight, of the total fatty acid content of the oil. In some embodiments, the oil contains less than about 0.5% DPAn6, by weight, of the total fatty acid content of the oil. In some embodiments, the oil does not contain any detectable amount of DPAn6. As used herein, an emulsion “substantially free of DPAn6” refers to an emulsion containing less than about 2% DPAn6, by weight, docosapentaenoic acid 22:5n-6 (DPAn6) of the total fatty acid content of the emulsion. In some embodiments, the emulsion contains less than about 1% DPAn6, by weight, of the total fatty acid content of the emulsion. In some embodiments, the emulsion contains less than about 0.5% DPAn6, by weight, of the total fatty acid content of the emulsion. In some embodiments, the emulsion does not contain any detectable amount of DPAn6.

The oil containing DHA, or emulsion containing DHA-FFA, can also be substantially free of arachidonic acid (ARA). ARA refers to the compound (all-Z)-5,8,11,14-eicosatetraenoic acid (also referred to as (5Z,8Z,11Z,14Z)-icosa-5,8,11,14-tetraenoic acid), as well as any salts or derivatives thereof. Thus, the term “ARA” encompasses the free acid ARA as well as ARA alkyl esters and triglycerides containing ARA. ARA is an ω-6 polyunsaturated fatty acid. As used herein, an oil “substantially free of ARA” refers to an oil in which ARA is less than about 3%, by weight, of the total fatty acid content of the oil. In some embodiments, the oil comprises, less than about 2% ARA, by weight, of the total fatty acid content of the oil, less than about 1% ARA, by weight, of the total fatty acid content of the oil, less than about 0.5% ARA, by weight, of the total fatty acid content of the oil, less than about 0.2% ARA, by weight, of the total fatty acid content of the oil, or less than about 0.01% ARA, by weight, of the total fatty acid content of the oil. In some embodiments, the oil has no detectable amount of ARA. As used herein, an emulsion “substantially free of ARA” refers to an emulsion in which ARA is less than about 3%, by weight, of the total fatty acid content of the emulsion. In some embodiments, the emulsion comprises, less than about 2% ARA, by weight, of the total fatty acid content of the emulsion, less than about 1% ARA, by weight, of the total fatty acid content of the emulsion, less than about 0.5% ARA, by weight, of the total fatty acid content of the emulsion, less than about 0.2% ARA, by weight, of the total fatty acid content of the emulsion, or less than about 0.01% ARA, by weight, of the total fatty acid content of the emulsion. In some embodiments, the emulsion has no detectable amount of ARA.

DHA-FFA can be obtained by hydrolyzing the ester bond of a DHA ester or triglyceride containing DHA, resulting in formation of the free fatty acid form of DHA. The DHA of the present invention can be derived from various sources, e.g., from oleaginous microorganisms. As used herein, “oleaginous microorganisms” are defined as microorganisms capable of accumulating greater than 20% of the dry weight of their cells in the form of lipids. In some embodiments, the DHA is derived from a phototrophic or heterotrophic single cell organism or multicellular organism, e.g., an algae. For example, the DHA can be derived from or initially derived from a diatom, e.g., a marine dinoflagellates (algae), such as Crypthecodinium sp., Thraustochytrium sp., Schizochytrium sp., or combinations thereof. The source of the DHA can include a microbial source, including the microbial groups Stramenopiles, Thraustochytrids, and Labrinthulids. Stramenopiles includes microalgae and algae-like microorganisms, including the following groups of microorganisms: Hamatores, Proteromonads, Opalines, Develpayella, Diplophrys, Labrinthulids, Thraustochytrids, Biosecids, Oomycetes, Hypochytridiomycetes, Commation, Reticulosphaera, Pelagomonas, Pelagococcus, Ollicola, Aureococcus, Parmales, Diatoms, Xanthophytes, Phaeophytes (brown algae), Eustigmatophytes, Raphidophytes, Synurids, Axodines (including Rhizochromulinaales, Pedinellales, Dictyochales), Chrysomeridales, Sarcinochrysidales, Hydrurales, Hibberdiales, and Chromulinales. The Thraustochytrids include the genera Schizochytrium (species include aggregatum, limnaceum, mangrovei, minutum, octosporum), Thraustochytrium (species include arudimentale, aureum, benthicola, globosum, kinnei, motivum, multirudimentale, pachydermum, proliferum, roseum, striatum), Ulkenia (species include amoeboidea, kerguelensis, minuta, profunda, radiate, sailens, sarkariana, schizochytrops, visurgensis, yorkensis), Aplanochytrium (species include haliotidis, kerguelensis, profunda, stocchinoi), Japonochytrium (species include marinum), Althomia (species include crouchii), and Elina (species include marisalba, sinorifica). The Labrinthulids include the genera Labyrinthula (species include algeriensis, coenocystis, chattonii, macrocystis, macrocystis atlantica, macrocystis macro cystis, marina, minuta, roscoffensis, valkanovii, vitellina, vitellina pacifica, vitellina vitellina, zopfi), Labyrinthomyxa (species include marina), Labyrinthuloides (species include haliotidis, yorkensis), Diplophrys (species include archeri), Pyrrhosorus* (species include marinus), Sorodiplophrys* (species include stercorea), and Chlamydomyxa* (species include labyrinthuloides, montana) (*=there is no current general consensus on the exact taxonomic placement of these genera).

In some embodiments, the algal source is, e.g., Crypthecodinium cohnii. Samples of C. cohnii, have been deposited with the American Type Culture Collection at Rockville, Md., and assigned accession nos. 40750, 30021, 30334-30348, 30541-30543, 30555-30557, 30571, 30572, 30772-30775, 30812, 40750, 50050-50060, and 50297-50300.

As used herein, the term microorganism, or any specific type of organism, includes wild strains, mutants or recombinant types. Organisms which can produce an enhanced level of oil containing DHA are considered to be within the scope of this invention. Also included are microorganisms designed to efficiently use more cost-effective substrates while producing the same amount of DHA as the comparable wild-type strains. Cultivation of dinoflagellates such as C. cohnii has been described previously. See, U.S. Pat. No. 5,492,938 and Henderson et al., Phytochemistry 27:1679-1683 (1988). Organisms useful in the production of DHA can also include any manner of transgenic or other genetically modified organisms, e.g., plants, grown either in culture fermentation or in crop plants, e.g., cereals such as maize, barley, wheat, rice, sorghum, pearl millet, corn, rye and oats; or beans, soybeans, peppers, lettuce, peas, Brassica species (e.g., cabbage, broccoli, cauliflower, brussel sprouts, rapeseed, and radish), carrot, beets, eggplant, spinach, cucumber, squash, melons, cantaloupe, sunflowers, safflower, canola, flax, peanut, mustard, rapeseed, chickpea, lentil, white clover, olive, palm, borage, evening primrose, linseed, and tobacco.

Another source of oils containing DHA suitable for the compositions and methods of the present invention includes an animal source. Examples of animal sources include aquatic animals (e.g., fish, marine mammals, and crustaceans such as krill and other euphausids) and animal tissues (e.g., brain, liver, eyes, etc.) and animal products such as eggs or milk. Thus, in some embodiments, the method of the present invention comprises administering daily to the subject an emulsion comprising DHA-FFA substantially free of eicosapentaenoic acid (EPA), wherein the DHA-FFA is obtained from DHA, and the DHA is derived from a non-algal source, e.g., fish.

DHA can be purified to various levels. DHA purification can be achieved by any means known to those of skill in the art, and can include the extraction of total oil from an organism which produces DHA. In some embodiments, EPA, ARA, DPAn6 and/or flavonoids are then removed from the total oil, for example, via chromatographic methods. Alternatively, DHA purification can be achieved by extraction of total oil from an organism which produces DHA, but produces little, if any, amount of EPA, ARA, DPAn6 and/or flavonoids. Similarly, DHA-FFA can be purified to various levels. For example, various purity levels of DHA-FFA can be obtained by using various purities of DHA as described herein. In some embodiments, the oil can be diluted with sunflower oil to achieve the desired concentration of fatty acids.

Microbial oils useful in the present invention can be recovered from microbial sources by any suitable means known to those in the art. For example, the oils can be recovered by extraction with solvents such as chloroform, hexane, methylene chloride, methanol and the like, or by supercritical fluid extraction. Alternatively, the oils can be extracted using extraction techniques, such as are described in U.S. Pat. No. 6,750,048 and International Pub. No. WO/2001/053512, both filed Jan. 19, 2001, both of which are incorporated herein by reference in their entirety.

Additional extraction and/or purification techniques are taught in International Pub. No. WO2001076715; International Pub. No. WO/2001/076385; U.S. Pub. No. 2007/0004678; U.S. Pub. No. 2005/0129739; U.S. Pat. No. 6,399,803; and International Pub. No. WO/2001/051598; all of which are incorporated herein by reference in their entirety. The extracted oils can be evaporated under reduced pressure to produce a sample of concentrated oil material. Processes for the enzyme treatment of biomass for the recovery of lipids are disclosed in International Pub. No. WO/2003/092628; U.S. Pub. No. 20050170479; EP Pat. Pub. 0776356 and U.S. Pat. No. 5,928,696, all of which are incorporated herein by reference in their entirety.

DHA can be prepared as esters using a method comprising: a) reacting a composition comprising polyunsaturated fatty acids in the presence of an alcohol and a base to produce an ester of a polyunsaturated fatty acid from the triglycerides; and b) distilling the composition to recover a fraction comprising the ester of the polyunsaturated fatty acid, optionally wherein the method further comprises: c) combining the fraction comprising the ester of the polyunsaturated fatty acid with urea in a medium; d) cooling or concentrating the medium to form a urea-containing precipitate and a liquid fraction; and e) separating the precipitate from the liquid fraction. See, e.g., U.S. patent publication no. US 2009/0023808, incorporated by reference herein in its entirety. In some embodiments, the purification process includes starting with refined, bleached, and deodorized oil (RBD oil), then performing low temperature fractionation using acetone to provide a concentrate. The concentrate can be obtained by base-catalyzed transesterification, distillation, and silica refining to produce the final DHA product. In some embodiments, DHA free fatty acids can be prepared using a method as described in U.S. Appl. No. TBD (Attorney Docket No. 2715.2570000), entitled “Method of preparing free polyunsaturated fatty acids” filed Feb. 18, 2011, incorporated herewith in its entirety.

Methods of determining purity levels of fatty acids are known in the art, and can include, e.g., chromatographic methods such as, e.g., HPLC silver ion chromatographic columns (ChromSpher 5 Lipids HPLC Column, Chrompack, Raritan N.J.). Alternatively, the purity level can be determined by gas chromatography, with or without converting DHA to the corresponding methyl ester.

In some embodiments, DHA esters can be derived from undiluted oil from a single cell microorganism described above, and in some embodiments, from undiluted DHASCO®-T (Martek Biosciences Corporation, Columbia, Md.). In some embodiments, the oil from which DHA of the invention are derived include single cell microorganism oils that are manufactured by a controlled fermentation process followed by oil extraction and purification using methods common to the vegetable oil industry. In certain embodiments, the oil extraction and purification steps include refining, bleaching, and deodorizing. In some embodiments, the undiluted DHA oil comprises about 40% to about 50% DHA by weight (about 400-500 mg DHA/g oil). In certain embodiments, the undiluted DHA oil is enriched by cold fractionation (resulting in oil containing about 60% w/w of DHA triglyceride), which DHA fraction optionally can be transesterified, and subjected to further downstream processing to produce the active DHA of the invention. In some embodiments of the invention, downstream processing of the oil comprises distillation and/or silica refinement.

Thus, to produce oil from which DHA of the invention is derived, in certain aspects of the invention, the following steps are used: fermentation of a DHA producing microorganism; harvesting the biomass; spray drying the biomass; extracting oil from the biomass; refining the oil; bleaching the oil; chill filtering the oil; deodorizing the oil; and adding an antioxidant to the oil. In some embodiments, the microorganism culture is progressively transferred from smaller scale fermenters to a production size fermenter. In some embodiments, following a controlled growth over a pre-established period, the culture is harvested by centrifugation then pasteurized and spray dried. In certain embodiments, the dried biomass is flushed with nitrogen and packaged before being stored frozen at −20° C. In certain embodiments, the DHA oil is extracted from the dried biomass by mixing the biomass with n-hexane or isohexane in a batch process which disrupts the cells and allows the oil and cellular debris to be separated. In certain embodiments, the solvent is then removed.

In some embodiments, the crude DHA oil then undergoes a refining process to remove free fatty acids and phospholipids. The refined DHA oil is transferred to a vacuum bleaching vessel to assist in removing any remaining polar compounds and pro-oxidant metals, and to break down lipid oxidation products. The refined and bleached DHA oil undergoes a final clarification step by chilling and filtering the oil to facilitate the removal of any remaining insoluble fats, waxes, and solids.

Optionally, the DHA is deodorized under vacuum in a packed column, counter current steam stripping deodorizer. Antioxidants such as ascorbyl palmitate and alpha-tocopherol can optionally be added to the deodorized oil to help stabilize the oil. In some embodiments, the final, undiluted DHA oil is maintained frozen at −20° C. until further processing.

In some embodiments, the DHA oil is converted to DHA ester by methods known in the art. In some embodiments, DHA esters of the invention are produced from DHA oil by the following steps: cold fractionation and filtration of the DHA oil (to yield for example about 60% triglyceride oil); direct transesterification (to yield about 60% DHA ethyl ester); molecular distillation (to yield about 88% DHA ethyl ester); silica refinement (to yield about 90% DHA ethyl ester); and addition of an antioxidant.

In some embodiments, the cold fractionation step is carried out as follows: undiluted DHA oil (triglyceride) at about 500 mg DHA/g oil is mixed with acetone and cooled at a controlled rate in a tank with −80° C. chilling capabilities. Saturated triglycerides crystallize out of solution, while polyunsaturated triglycerides at about 600 mg DHA/g oil remain in the liquid state. The solids containing about 300 mg DHA/g oil are filtered out with a 20 micron stainless steel screen from the liquid stream containing about 600 mg DHA/g oil. The solids stream is then heated (melted) and collected. The 600 mg DHA/g oil liquid stream is desolventized with heat and vacuum and then transferred to the transesterification reactor.

In some embodiments, the transesterification step is carried out on the 600 mg/g DHA oil, wherein the transesterification is done via direct transesterification using ethanol and sodium ethoxide. The transesterified material DHA ethyl ester (“DHA-EE”) is then subject to molecular distillation and thus, further distilled (3 passes, heavies, lights, heavies) to remove most of the other saturated fatty acids and some sterols and non-saponifiable material. The DHA-EE is further refined by passing it through a silica column.

DHA free fatty acids can be made using, for example, the DHA containing oils described above. In some embodiments, the DHA-FFA can be obtained from DHA esters. DHA triglycerides, for example, can be saponified followed by a urea adduction step to make free fatty acids. An non-limiting example for making a DHA free fatty acid oil is provided below in the Example section.

Additional fatty acids can be present in the oil and/or the emulsion. These fatty acids can include fatty acids that are not removed during the purification process, i.e., fatty acids that are co-isolated with DHA from an organism. These fatty acids can be present in various concentrations. In some embodiments, the oil comprises 0.1% to 60% of one or more of the following fatty acids, or esters thereof: (a) capric acid; (b) lauric acid; (c) myristic acid; (d) palmitic acid, (e) palmitoleic acid; (f) stearic acid; (g) oleic acid; (h) linoleic acid; (i) α-linolenic acid; (j) docosapentaenoic acid 22:5n-3, 22:5w3 (DPAn3); and (k) 4,7,10,13,16,19,22,25-octacosaoctaenoic acid (C28:8). In some embodiments, the oil comprises 20% to 40% of one or more of the following fatty acids, or esters thereof: (a) capric acid; (b) lauric acid; (c) myristic acid; (d) palmitic acid; (e) palmitoleic acid; (f) stearic acid; (g) oleic acid; (h) linoleic acid; (i) α-linolenic acid; U) docosapentaenoic acid 22:5n-3, 22:5w3 (DPAn3); and (k) 4,7,10,13,16,19,22,25-octacosaoctaenoic acid (C28:8). In some embodiments, the oil comprises less than about 1% each of the following fatty acids, or esters thereof: (a) capric acid; (b) lauric acid; (c) myristic acid; (d) palmitic acid, (e) palmitoleic acid; (f) stearic acid; (g) oleic acid; (h) linoleic acid; (i) α-linolenic acid; (j) docosapentaenoic acid 22:5n-3, 22:5w3 (DPAn3); and (k) 4,7,10,13,16,19,22,25-octacosaoctaenoic acid (C28:8).

In some embodiments, an oil is characterized by a fatty acid content of about 0.1% to about 20% (w/w) of one or more of the following fatty acids or esters thereof: (a) capric acid; (b) lauric acid; (c) myristic acid; (d) palmitic acid; (e) palmitoleic acid; (f) stearic acid; (g) oleic acid; (h) linoleic acid; (i) α-linolenic acid; (j) docosapentaenoic acid 22:5n-3, 22:5w3 (DPAn3); and (k) 4,7,10,13,16,19,22,25-octacosaoctaenoic acid (C28:8). As used herein, the terms “or less” or “less than about” refers to percentages that include 0%, or amounts not detectable by current means. As used herein, “max” refers to percentages that include 0%, or amounts not detectable by current means.

In some embodiments, an oil is characterized by a fatty acid content of about 1.0% to about 5% (w/w) of one or more of the following fatty acids or esters thereof: (a) capric acid; (b) lauric acid; (c) myristic acid; (d) palmitic acid; (e) palmitoleic acid; (f) stearic acid; (g) oleic acid; (h) linoleic acid; (i) α-linolenic acid; (j) docosapentaenoic acid 22:5n-3, 22:5w3 (DPAn3); and (k) 4,7,10,13,16,19,22,25-octacosaoctaenoic acid (C28:8).

In some embodiments, an oil is characterized by a fatty acid content of less than about 1% (w/w) each of the following fatty acids or esters thereof: (a) capric acid; (b) lauric acid; (c) myristic acid; (d) palmitic acid; (e) palmitoleic acid; (f) stearic acid; (g) oleic acid; (h) linoleic acid; (i) α-linolenic acid; (j) docosapentaenoic acid 22:5n-3, 22:5w3 (DPAn3); (k) docosapentaenoic acid 22:5n-6, 22:5w6 (DPAn6); and (1) 4,7,10,13,16,19,22,25-octacosaoctaenoic acid (C28:8). In some embodiments, the oil of the present invention does not contain a detectable amount of docosapentaenoic acid 22:5n-3, 22:5w3 (DPAn3); docosapentaenoic acid 22:5n-6, 22:5w6 (DPAn6); and/or 4,7,10,13,16,19,22,25-octacosaoctaenoic acid (C28:8); of the total fatty acid content of the oil or unit dose.

In some of embodiments an oil is characterized by one or more the following fatty acids (or esters thereof), expressed as wt % of the total fatty acid content. The embodiments provided herein can further comprise about 2% or less (w/w) of capric acid (C10:0). The embodiments herein can further comprise about 6% or less (w/w) of lauric acid (C12:0). The embodiments herein can further comprise about 20% or less, or about 5 to about 20% (w/w) of myristic acid (C14:0). The embodiments herein can further comprise about 20% or less, or about 5% to about 20% (w/w) of palmitic acid (C16:0). The embodiments herein can further comprise about 3% or less (w/w) of palmitoleic acid (C16:1n-7). The embodiments herein can further comprise about 2% or less (w/w) of stearic acid (C18:0). The embodiments herein can further comprise about 40% or less, or about 10% to about 40% (w/w) of oleic acid (C18:1n-9). The embodiments herein can further comprise about 5% or less (w/w) of linoleic acid (C18:2). The embodiments herein can further comprise about 2% or less (w/w) of nervonic acid (C24:1). The embodiments herein can further comprise about 3% or less (w/w) of other fatty acids or esters thereof. An oil with the preceding characteristics can include DHASCO® (Martek Biosciences, Columbia, Md.), an oil derived from Crypthecodinium cohnii containing docosahexaenoic acid (DHA).

An exemplary DHA (triglyceride) containing oil derived from Crypthecodinium cohnii is characterized by the specified amount of components listed in Table 1, where “Max” refers to the amount of the component that can be present up to the specified amount.

TABLE 1

| | Concentration (wt/wt) |
|---|---|
| Fatty Acids | |
| 10:0 | Max 2% |
| 12:0 | Max 6% |
| 14:0 | 5%-20% |
| 16:0 | 5%-20% |
| 16:1 | Max 3% |
| 18:0 | Max 2% |
| 18:1 | 10%-40% |
| 18:2 | Max 5% |
| 22:6 DHA | 40% to 45% |
| 24:1 | Max 2% |
| Others | Max 3% |
| Elemental Composition | |
| Arsenic | Max 0.5 ppm |
| Copper | Max 0.1 ppm |
| Iron | Max 0.5 ppm |
| Lead | Max 0.2 ppm |
| Mercury | Max 0.04 ppm |
| Phosphorous | Max 10 ppm |
| Chemical Characteristics | |
| Peroxide value | Max 5 meq/kg |
| Free fatty acid | Max 0.4% |
| Unsaponifiable Matter | Max 3.5% |

An exemplary undiluted DHA (triglyceride) containing oil derived from Crypthecodinium cohnii is characterized by amount of DHA described herein, and one or more, or all of the features listed below in Table 2, where “Max” refers to the amount of the component that can be present up to the specified amount.

TABLE 2
Characteristics of Undiluted DHA Oil

| Test | Specification |
|---|---|
| DHA content mg/DHA/g oil | Min 480 mg/g |
| Free Fatty Acid | Max. 0.4% |
| Peroxide Value (PV) | Max. 5 meq/kg |
| Anisidine Value (AV) | Max 20 |
| Moisture and Volatiles (M & V) | Max. 0.02% |
| Unsaponifiable Matter | Max. 3.5% |
| Insoluble Impurities | Max. 0.1% |
| Trans Fatty Acid | Max. 1% |
| Arsenic | Max. 0.5 ppm |
| Cadmium | Max. 0.2 ppm |
| Chromium | Max. 0.2 ppm |
| Copper | Max. 0.1 ppm |
| Iron | Max. 0.5 ppm |
| Lead | Max. 0.2 ppm |
| Manganese | Max. 0.04 ppm |
| Mercury | Max. 0.04 ppm |
| Molybdenum | Max. 0.2 ppm |
| Nickel | Max. 0.2 ppm |
| Phosphorus | Max. 10 ppm |
| Silicon | Max. 500 ppm |
| Sulfur | Max. 100 ppm |
| 18:1 n-9 Oleic Acid | Max. 10% |
| 20:5 n-3 EPA | Max. 0.1% |
| Unknown Fatty Acids | Max. 3.0% |

In some embodiments, an oil is characterized by one or more the following fatty acids (or esters thereof), expressed as wt % of the total fatty acid content. The embodiments provided herein can further comprise about 2% or less (w/w) of capric acid (C10:0). The embodiments provided herein can further comprise about 6% or less (w/w) of lauric acid (C12:0). The embodiments provided herein can further comprise about 20% or less, or about 10 to about 20% (w/w) of myristic acid (C14:0). The embodiments provided herein can further comprise about 15% or less, or about 5 to about 15% (w/w) of palmitic acid (C16:0). The embodiments provided herein can further comprise about 5% or less (w/w) of palmitoleic acid (C16:1n-7). The embodiments provided herein can further comprise about 2% or less (w/w) of stearic acid (C18:0). The embodiments provided herein can further comprise about 20% or less, or about 5% to about 20% (w/w) of oleic acid (C18:1n-9). The embodiments provided herein can further comprise about 2% or less (w/w) of linoleic acid (C18:2). The embodiments provided herein can further comprise about 2% or less (w/w) of nervonic acid (C24:1). The embodiments provided herein can further comprise about 3% or less (w/w) of other fatty acids. An oil with the preceding characteristics can be an oil derived from Crypthecodinium cohnii containing docosahexaenoic acid (DHA).

An exemplary DHA containing oil derived from Crypthecodinium cohnii is characterized by the specified amount of components listed in Table 3, where “Max” refers to the amount of the component that can be present up to the specified amount.

TABLE 3

| | Concentration (wt/wt) |
|---|---|
| Fatty Acids | |
| 10:0 | 0-2% |
| 12:0 | 0-6% |
| 14:0 | 10%-20% |
| 16:0 | 5%-15% |
| 16:1 | 0-5% |
| 18:0 | 0-2% |
| 18:1 | 5%-20% |
| 18:2 | 0-2%% |
| 22:6 n-3 DHA | 57%-65% |
| 24:1 | 0-2% |
| Others | 0-3% |
| Elemental Composition | |
| Arsenic | Max 0.5 ppm |
| Copper | Max 0.1 ppm |
| Iron | Max 0.5 ppm |
| Lead | Max 0.2 ppm |
| Mercury | Max 0.2 ppm |
| Phosphorous | Max 10 ppm |
| Chemical Characteristics | |
| Peroxide value | Max 5 meq/kg |
| Free fatty acid | Max 0.4% |
| Unsaponifiable Matter | Max 3.5% |
| Trans fatty acids | <3.5% |
| Moisture and Volatiles | <0.1% |
| Insoluble impurities | <0.1% |

In some embodiments and oil is characterized by one or more the following fatty acids (or esters thereof), expressed as wt % of the total fatty acid content: The embodiments provided herein can further comprise about 0.1% or less (w/w) of myristic acid (C14:0) or is not detectable. The embodiments provided herein can further comprise about 0.5% or less (w/w) of palmitic acid (C16:0). The embodiments provided herein can further comprise about 0.5% or less (w/w) of palmitoleic acid (C16:1n-7). The embodiments provided herein can further comprise about 0.5% or less (w/w) of stearic acid (C18:0), or is not detectable. The embodiments provided herein can further comprise about 4% or less (w/w) of oleic acid (C18:1n-9). The embodiments provided herein can further comprise less than 0.1% (w/w) of linoleic acid (C18:2) or is not detectable. The embodiments provided herein can further comprise less than 0.1% (w/w) of eicosapentaenoic acid (C20:5) or is not detectable. The embodiments provided herein can further comprise about 2% or less (w/w) of decosapentaenoic acid (22:5n-3). The embodiments provided herein can further comprise about 1% or less (w/w) of octacosaoctaenoic acid (28:8 n-3). The embodiments provided herein can further comprise about 0.5% or less (w/w) of tetracosaenoic acid (24:1n9). The embodiments provided herein can further comprise about 1% or less (w/w) of other fatty acids. The DHA in oil with the preceding characteristics can be in the form of a DHA ester, preferably an alkyl ester, such as a methyl ester, ethyl ester, propyl ester, or combinations thereof, prepared from an algal oil prepared from the Crypthecodinium, cohnii sp.

An exemplary DHA-containing oil derived from the algal oil of Crypthecodinium Cohnii, wherein the DHA comprises an ethyl ester, can be characterized by the specified amount of components listed in Table 4, where “Max” refers to the amount of the component that can be present up to the specified amount.

TABLE 4

| | |
|---|---|
| DHA content (mg/g) | 855-945 |
| Fatty Acid Content: % of total EE | |
| Eicosapentaenoic Acid (20:5ω3) | ND |
| Myristic Acid (14:0) | 0.1% |
| Palmitic Acid (16:0) | 0.5% |
| Palmitoleic Acid (16:1ω7) | 0.4% |
| Stearic Acid (18:0) | ND |
| Oleic Acid (18:1ω9) | 4% |
| Linoleic Acid (18:2ω6) | ND |
| Docosapentaenoic acid (22:5ω3) | 1.3% |
| Octacosaoctaenoic acid (28:8ω3) | 0.9% |
| Tetracosaenoic Acid (24:1ω9) | 0.3% |
| Others | 1.1% |
| Elemental Composition | |
| Arsenic | Max 0.5 ppm |
| Copper | Max 0.1 ppm |
| Iron | Max 0.5 ppm |
| Lead | Max 0.2 ppm |
| Mercury | Max 0.04 ppm |
| Chemical Characteristics | |
| Peroxide value | Max 10.0 meq/kg |

ND = not detectable

In some embodiments of the oil is characterized by one or more the following fatty acids (or esters thereof), expressed as wt % of the total fatty acid content. The embodiments provided herein can further comprise about 12% or less, or about 6% to about 12% (w/w) of myristic acid (C14:0). The embodiments provided herein can further comprise about 28% or less, or about 18 to about 28% (w/w) of palmitic acid (C16:0). The embodiments provided herein can further comprise about 2% or less (w/w) of stearic acid (C18:0). The embodiments provided herein can further comprise about 8% or less of (w/w) oleic acid (C18:1n-9). The embodiments provided herein can further comprise about 2% or less (w/w) of linoleic acid (C18:2). The embodiments provided herein can further comprise about 2% or less (w/w) of arachidonic acid (C20:4). The embodiments provided herein can further comprise about 3% or less (w/w) of eicosapentaenoic acid (C20:5). The embodiments provided herein can further comprise about 18% or less, or about 12% to about 18% (w/w) of decosapentaenoic acid (22:5n-6). The embodiments provided herein can further comprise about 10% or less (w/w) of other fatty acids. In some of these embodiments, the ratio of wt % of DHA to wt % of DPAn6 is about 2.5 to about 2.7. An oil with the preceding characteristics can comprise Life's DHA™ (also formerly referenced as DHA-S and DHASCO®), Martek Biosciences, Columbia, Md.), an oil derived from the Thraustochytrid, Schizochytrium sp., that contains a high amount of DHA and also contains docosapentaenoic acid (n-6) (DPAn-6).

An exemplary DHA (triglyceride) containing oil derived from Schizochytrium sp. is characterized by the specified amount of components listed in Table 5, where “Max” refers to the amount of the component that can be present up to the specified amount.

TABLE 5

| | Concentration (wt/wt) |
|---|---|
| Fatty Acids | |
| 14:0 | 6.0%-12.0% |
| 16:0 | 18%-28% |
| 18:0 | Max 2% |
| 18:1 | Max 8% |
| 18:2 | Max 2% |
| 20:4 ARA | Max 2% |
| 20:5 EPA | Max 3% |
| 22:5n-6 DPA | 12%-18% |
| 22:6 DHA | Min 35% |
| Others | Max 10% |
| Elemental Composition | |
| Arsenic | Max 0.2 ppm |
| Copper | Max 0.05 ppm |
| Iron | Max 0.2 ppm |
| Lead | Max 0.1 ppm |
| Mercury | Max 0.04 ppm |
| Chemical Characteristics | |
| Peroxide value | Max 5 meq/kg |
| Free fatty acid | Max 0.25% |
| Moisture and Volatiles | Max 0.05% |
| Unsaponifiable Matter | Max 4.5% |
| Trans fatty acids | Max 1% |

The DHA in an oil can be in the form of a DHA ester, preferably an alkyl ester, such as a methyl ester, ethyl ester, propyl ester, or combinations thereof, prepared from an algal oil prepared from derived from the Thraustochytrid, Schizochytrium sp. An exemplary DHA (ethyl esters) containing oil derived from Schizochytrium sp. is characterized by the specified amount of components listed in Table 4 of WO 2009/006317, incorporated by reference herein. In some of these embodiments, an oil comprises DHA≧than about 57% (w/w), particularly ≧about 70% (w/w) of the total fatty acid content of the oil or unit dose. In some of these embodiments, the ratio of wt % of DHA to wt % of DPAn6 is about 2.5 to about 2.7.

An exemplary DHA (free fatty acid) containing oil is characterized by the specified amount of components listed in Table 6:

TABLE 6

| | Concentration (wt/wt) |
|---|---|
| Fatty Acids | |
| 10:0 | Max 0.5% |
| 12:0 | Max 0.5% |
| 14:0 | Max 0.5% |
| 14:1 | Max 0.5% |
| 16:0 | Max 0.5% |
| 16:1 | Max 0.5% |
| 18:1 (n-9) | Max 0.5% |
| 20:5 (n-3) EPA | Max 0.5% |
| 22:5 (n-3) DPA | Max 1% |
| 22:6 (n-3) DHA | Min 95% |
| 28:8 | Max 1.5% |
| Chemical Characteristics | |
| Docosahexaenoic acid | 946 mg/g |
| Docosahexaenoic acid | 98% |
| Free Fatty Acids | 93% |
| Trans Fatty Acids | <1% |

The following examples are for illustrative purposes and are not meant to be limiting.

### EXAMPLES

### Example 1

### Production of Free Fatty Acid

### a) Saponification

To 203 kg of ethanol, 13.5 kg of distilled water and 45.5 kg of potassium hydroxide was slowly added to 90 kg of 40-60% DHA triglyceride oil at room temperature while stifling. The reaction suspension was allowed to stir for a minimum of 2 hours between 40-50° C. After 2 hrs of reaction, the temperature of the reaction was lowered to room temperature. To the cooled reaction solution was slowly added 258 kg of the distilled water. To the diluted solution was then added 240 kg of iso-hexane. When phases were separated, the organic layer was removed. The aqueous layer was washed with 4×120 kg of iso-hexane. The aqueous layer was then acidified with a solution of 10N sulfuric acid. The acidified fatty acids were then extracted with 2×125 kg of iso-hexane. The combined organic layer was washed with a 4.8% sodium sulfate solution four times. The washed organic layer was dried over anhydrous sodium sulfate and concentrated to obtain 98.2 kg of light yellow free fatty acids oil.

### b) Urea Adduction

To 1,080 kg of methanol at a temperature greater than 60° C. was added 393 kg of urea portion-wise while stirring. When all urea was dissolved, 98.2 kg of light yellow free fatty acids oil (heated at between 65-70° C.) from Step a) above was slowly added to the urea solution. Once the oil has been added to the urea solution, the solution was slowly cooled to 10° C. with a mild agitation. When the temperature of the reaction solution reached 10° C., the agitation was stopped. The reaction solution was left overnight without stirring. The complexed crystals between urea and saturated fatty acids formed overnight. Next day, the filtrate was separated from the crystals. To the filtrate was then added 491 kg of the distilled water and the solution was allowed to stir at room temperature. While stirring, pH of the methanolic solution was then adjusted to 1.0 with a solution of 10N sulfuric acid. To the acidic solution was then added 131 kg of iso-hexane. The mixture was allowed to stir for 15 min. When phases were separated, the organic layer was separated and saved. The aqueous layer was extracted with another 131 kg of iso-hexane. The combined organic layer was washed with 164 kg of a 4.8% sodium sulfate solution three times and 196 kg of the distilled water. The washed organic layer was dried over anhydrous sodium sulfate and concentrated to obtain 46.5 kg of light yellow oil as >85% pure DHA free fatty acid oil.

### c) Distillation

20.6 Kg of the urea adducted DHA free fatty acid oil from step b) above was distilled using a wipe-film evaporator (Pope Scientific). The distillation involved three stages; 1. degassing; the processing conditions are as follows (temperature, 130-160° C.; pressure, <1.0 torr; feed pump setting and feed rate, 40-60 and 6-9 liter/hr; molecular still wiper rotation, 175-200 rpm) 2. long chain fatty acid, “Lights,” removal; the processing conditions are as follows (temperature, 150-180° C.; pressure, <0.1 torr; feed pump setting and feed rate, 10-60 and 2-9 liter/hr; molecular still wiper rotation, 175-200 rpm) 3. The “heavies” removal, i.e., longer chain fatty acids>C22 was performed as follows (temperature, 175-209° C.; pressure, <0.1 torr, feed pump setting and feed rate, 20-60 and 3-9 liters/hr; molecular still wiper rotation, 175-200 rpm). The lights removal, i.e., shorter chain fatty acids<C22, was usually performed three times before going into the Heavies removal. After the final Heavies removal, 9.51 kg of the DHA free fatty acid oil, with a >95% purity, was obtained as a light pale yellow oil.

### Example 2

Using a Silverson high shear mixer, 216 gm of Lipoid E 80 SN was dispersed while still frozen in 648 ml of distilled water (nitrogen protected) with the temperature of water for injection used being between 65-90° C. under nitrogen. The dispersion was continued under a blanket of nitrogen until Lipoid E 80 SN is finely divided and a viscous fluid is formed. 300 gm of glycerin was added while continuing the dispersion under a blanket of nitrogen. The distilled water (nitrogen protected, between 65-90° C.) was added to bring the total volume to 1,296 ml. The diluted Lipoid E 80 SN/glycerin dispersion was then passed through a homogenizer (Niro Soavi NS1001L2K) at ˜5,000 psi for a time equivalent to 10 continuous discrete passes. The dispersion in the reservoir was continuously stirred with an overhead stirrer under a blanket of nitrogen. After the homogenization, pH of the dispersion was adjusted to 9.0 with a solution of 0.5N sodium hydroxide, to obtain 1,754 gm of almost transparent light tan Lipoid E80 SN/glycerin dispersion.

To the pH adjusted Lipoid E80 SN/glycerin dispersion (146 gm, one twelfth of the dispersion from Example 2) at 40-75° C. was added to a thin stream of 300 gm of a DHA free fatty acid oil (contains >95% pure DHA free fatty acid) that has been previously heated to 70° C., while dispersing using a Silverson high shear mixer under a blanket of nitrogen. After 10 min, to this dispersion was added an additional 73 gm (one twenty-fourth of the dispersion from Example 2 of Lipoid E80 SN/glycerin dispersion while dispersing using a Silverson high shear mixer under a blanket of nitrogen. After additional 10 minutes of mixing, an additional 73 gm (one twenty-fourth of the dispersion from Example 2 of Lipoid E80 SN/glycerin dispersion was added, while dispersing using the mixer under a blanket of nitrogen. A total of 292 gm of the dispersion from Example 2 was used in this experiment. The distilled water (nitrogen protected, between 65-90° C.) was added to bring the total volume to ˜1,900 ml. The coarse emulsion was then passed through a homogenizer (Niro Soavi NS1001L2K) at ˜10,000 psi for a time equivalent to 10 continuous discrete passes at temperatures between 50-70° C. The dispersion in the reservoir was continuously stirred with an overhead stirrer under a blanket of nitrogen. The estimated final was about 2 L. A white lipid emulsion resulted, and the mean particle size of lipid emulsion was measured using a Malvern Mastersizer 2000. See Table 7.

TABLE 7
Instrument settings

| | | | |
|---|---|---|---|
| Accessory | Hydor 2000S | Obscuration | 15.04% |
| Name | | | |
| Analysis | General purpose | Dispersant | Water |
| model | | name | |
| Sensitivity | Enhanced | Dispersant RI | 1.330 |
| Particle RI | 1.390 | Weighted | 2.657% |
| | | Residual | |
| Absorption | 0.001 | Result | Off |
| | | Emulation | |
| Size Range | 0.020 to 2000.000 μm | | |

Sample Characteristics

| | | | | |
|---|---|---|---|---|
| Concentration | 0.1967% vol | Specific | 50.7 | m²/g |
| | | Surface Area | | |
| Span | 1.066 | Surface | 0.118 | μm |
| | | Weighted Mean | | |
| Uniformity | 0.331 | Vol | 0.138 | μm |
| | | Weighted Mean | | |
| Results Units | Volume | d(0.1): | 0.075 | μm |
| | | d(0.5): | 0.129 | μm |
| | | d(0.9): | 0.213 | μm |

| | | | |
|---|---|---|---|
| DHA potency | 139.2 mg/mL | PFAT5 | 3.360% |
| Oil/solid | 15.0% | pH | 3.88 |
| percentage | | | |

The pH was adjusted to about 8.02 with a solution of 0.5N sodium hydroxide (NaOH) and the mean particle size of the emulsion was measured as above. See Table 8.

TABLE 8
Instrument Settings

| | | | |
|---|---|---|---|
| Accessory | Hydor 2000S | Obscuration | 16.87% |
| Name | | | |
| Analysis | General purpose | Dispersant | Water |
| model | | name | |
| Sensitivity | Enhanced | Dispersant RI | 1.330 |
| Particle RI | 1.390 | Weighted | 2.660% |
| | | Residual | |
| Absorption | 0.001 | Result | Off |
| | | Emulation | |
| Size Range | 0.020 to 2000.000 μm | | |

Sample Characteristics

| | | | | |
|---|---|---|---|---|
| Concentration | 0.2183% Vol | Specific | 49.8 | m²/g |
| | | Surface Area | | |
| Span | 1.083 | Surface | 0.120 | μm |
| | | Weighted Mean | | |
| Uniformity | 0.335 | Vol | 0.141 | μm |
| | | Weighted Mean | | |
| Results Units | Volume | d(0.1) | 0.076 | μm |
| | | d(0.5) | 0.132 | μm |
| | | d(0.9) | 0.219 | μm |

| | | | |
|---|---|---|---|
| DHA Potency | 115.7 mg/g | pH | 8.02 |
| Oil/solid | 14.06 g/100 ml | PFAT5 | 0.300% |
| percentage | | | |

### Example 3

Using a Silverson high shear mixer, 72 gm of Lipoid E 80 SN was dispersed while still frozen in 216 ml of the distilled water (nitrogen protected) with the temperature of water for injection used being between 65-90° C. under nitrogen. The dispersion was continued under a blanket of nitrogen until Lipoid E 80 SN is finely divided and a viscous fluid is formed. 102 gm of glycerin was added while continuing the dispersion under a blanket of nitrogen. The distilled water (nitrogen protected, between 65-90° C.) was added to bring the total volume to 430 ml. The diluted Lipoid E 80 SN/glycerin dispersion was then passed through a homogenizer (Niro Soavi NS1001L2K) at ˜5,000 psi for a time equivalent to 10 continuous discrete passes. The dispersion in the reservoir was continuously stirred with an overhead stirrer under a blanket of nitrogen. After the homogenization, pH of the dispersion was adjusted to 10.0 with a solution of 0.5N sodium hydroxide, to obtain almost transparent light tan Lipoid E80 SN/glycerin dispersion.

To the pH adjusted Lipoid E80 SN/glycerin dispersion (189 gm, from Example 5) at 40-75° C. was added a thin stream of 150 gm of a DHA free fatty acid oil (contains >95% pure DHA free fatty acid) that has been previously heated to 70° C., while dispersing using a Silverson high shear mixer under a blanket of nitrogen. The volume of dispersion was adjusted to approximately 500 ml during dispersing. The dispersing was continued for 20 min. The coarse emulsion was then passed through a homogenizer (Niro Soavi NS1001L2K) at ˜10,000 psi for a time equivalent to 10 continuous discrete passes at temperatures between 50-70° C. The dispersion in the reservoir was continuously stirred with an overhead stirrer under a blanket of nitrogen. A white lipid emulsion resulted, and the mean particle size of lipid emulsion was measured using a Malvern Mastersizer 2000.

The mean particle size of lipid emulsion was measured using a Malvern Mastersizer 2000. See Table 9.

TABLE 9
Instrument Settings

| | | | |
|---|---|---|---|
| Accessory | Hydor 2000S | Obscuration | 15.75% |
| Name | | | |
| Analysis | General purpose | Dispersant | Water |
| model | | name | |
| Sensitivity | Enhanced | Dispersant RI | 1.330 |
| Particle RI | 1.390 | Weighted | 2.572% |
| | | Residual | |
| Absorption | 0.001 | Result | Off |
| | | Emulation | |
| Size Range | 0.020 to 2000.000 μm | | |

Sample Characteristics

| | | | | |
|---|---|---|---|---|
| Concentration | 0.1998% Vol | Specific | 56.6 | m²/g |
| | | Surface Area | | |
| Span | 1.349 | Surface | 0.106 | μm |
| | | Weighted Mean | | |
| Uniformity | 0.423 | Vol | 0.137 | μm |
| | | Weighted Mean | | |
| Results Units | Volume | d(0.1) | 0.065 | μm |
| | | d(0.5) | 0.124 | μm |
| | | d(0.9) | 0.232 | μm |

| | | | |
|---|---|---|---|
| DHA potency | 224.2 mg/ml | pH | 5.45 |
| Oil/solid | 26.7 g/100 ml | PFAT5 | 0.283 |
| percentage | | | |

The pH of the emulsion (350 ml) was adjusted to ca. 8.0 with a solution of 0.5N sodium hydroxide about two days after the preparation outline above. The final volume of pH-adjusted emulsion reached 0.5 liter. Then the pH-adjusted emulsion was aliquoted into 20 ml Type 1/glass vials (15 ml/vial). The aliquot samples were flushed with nitrogen and sealed with rubber septums and aluminum seals. The sealed samples were autoclaved at 122° C. for 15 min. Finally the pH, D[4,3], and uniformity of the final emulsion were measured again. A sample emulsion was lyophilized to provide an oil-like sample. The oil sample was further analyzed for the DHA potency. See Table 10.

TABLE 10
Instrument Settings

| | | | |
|---|---|---|---|
| Accessory | Hydor 2000S | Obscuration | 17.08% |
| Name | | | |
| Analysis | General purpose | Dispersant | Water |
| model | | name | |
| Sensitivity | Enhanced | Dispersant RI | 1.330 |
| Particle RI | 1.390 | Weighted | 2.245% |
| | | Residual | |
| Absorption | 0.001 | Result | Off |
| | | Emulation | |
| Size Range | 0.020 to 2000.000 μm | | |

Sample Characteristics

| | | | | |
|---|---|---|---|---|
| Concentration | 0.2236% Vol | Specific | 53.4 | m²/g |
| | | Surface Area | | |
| Span | 1.206 | Surface | 0.112 | μm |
| | | Weighted Mean | | |
| Uniformity | 0.377 | Vol | 0.137 | μm |
| | | Weighted Mean | | |
| Results Units | Volume | d(0.1) | 0.070 | μm |
| | | d(0.5) | 0.126 | μm |
| | | d(0.9) | 0.221 | μm |

| | | | |
|---|---|---|---|
| DHA potency | 155.4 | pH | 8.09 |
| Oil/solid | 19.39 g/100 ml | PFAT5 | 0.208% |
| percentage | | | |

After the pH is adjusted to about 8.0 or above, the mean diameter and the uniformity measurements did not significantly change for at least 4 months in emulsions made according to the procedures specified above.

### Example 4

Frozen Lipoid E 80 SN (324 g) was added to 200 ml distilled water while stirring with a Silverson high shear mixer at temperatures between 65-90° C. under a nitrogen. The mixing was continued until Lipoid E 80 SN was finely divided and a viscous fluid was formed. Then to the mixture was added 300 g of glycerin portion wise. Additional distilled water was added to bring the total volume to 1300 ml. The diluted mixture was then transferred to a homogenizer (Niro Soavi NS1001L2K). The mixture was continuously passed through the homogenizer at 5,000 psi (ca. 350 bars) for a time equivalent to 10 discrete passes while maintaining the temperature at around 70° C. and stirring the retained mixture with an overhead stirrer under a nitrogen atmosphere. After the homogenization, the dispersion was filtered over 0.45 micron membrane filters. The pH of the filtered dispersion was adjusted to ca. 10.0 with a solution of 0.5N sodium hydroxide. At this point, the dispersion (2400 g) thus prepared was intended for 12 liters of final lipid emulsions.

Oil containing DHA (Table 6; >95% free fatty acid) was preheated at 70° C. To 300 g of the dispersion prepared above stifling with a Silverson high shear mixer at temperatures between 40-75° C. under a nitrogen atmosphere was added a thin stream of 450 g of the preheated DHA free fatty acid oil. The distilled water was used to rinse the containers. At this point, the combined volume of the dispersion was at 70% of the final intended volume. The mixture was allowed to stir at a high shear for 20 min. The coarse emulsion formed was then transferred to a homogenizer (Niro Soavi NS1001L2K). The containers were rinsed with distilled water to allow the combined coarse emulsion to reach a total volume of 1.2 liter. The emulsion was continuously passed through the homogenizer at 10,000 psi (ca 690 bars) for a time equivalent to 10 discrete passes while maintaining the temperature at around 70° C. and stifling the retained emulsion with an overhead stirrer under a nitrogen atmosphere. During the homogenization process, the pH and particle size distributions (mean diameter size (D[4,3]), and uniformity) of the emulsion were monitored with a pH meter and Malvern MasterSizer 2000. Upon completion of the homogenization, a white lipid emulsion was obtained and weighed. The pH of the emulsion (240 ml) was adjusted to ca. 8.0 with a solution of 1.0 N sodium hydroxide. Then the pH-adjusted emulsion was aliquoted into 20 ml Type 1/glass vials (15 ml/vial). The aliquot samples were flushed with nitrogen and sealed with chlorobutyl rubber stoppers and aluminum seals. The sealed samples were autoclaved at 122° C. for 15 min. Finally the pH, D[4,3], and uniformity of the final emulsion were measured again. A sample emulsion was lyophilized to provide an oil-like sample. The oil sample was further analyzed for the DHA potency (Table 11)

TABLE 11
Instrument Settings

| | | | |
|---|---|---|---|
| Accessory | Hydro 2000S | Obscuration | 15.74% |
| name | | | |
| Analysis | General purpose | Dispersant | Water |
| Model | | name | |
| Sensitivity | Enhanced | Dispersant RI | 1.330 |
| Particle RI | 1.390 | Weighted | 2.705% |
| | | Residual | |
| Absorption | 0.001 | Result | Off |
| | | Emulation | |
| Size Range | 0.020-2000.000 μm | | |

Sample Characteristics (TX-1598-80)

| | | | | |
|---|---|---|---|---|
| Concentration | 0.1111% Vol | Specific | 27.8 | m²/g |
| | | Surface Area | | |
| Span | 1.067 | Surf. Weighted | 0.216 | μm |
| | | Mean D[3,2] | | |
| Uniformity | 0.330 | Vol. Weighted | 0.255 | μm |
| | | Mean D[4, 3] | | |
| Results Units | Volume | d(0.1) | 0.136 | μm |
| | | d(0.5) | 0.242 | μm |
| | | d(0.9) | 0.394 | μm |

| | | | |
|---|---|---|---|
| DHA Potency | 236.1 mg/ml | pH | 8.05 |
| Oil/solid | 30.0 g/100 ml | PFAT5(%) | Not available |
| percentage | | | |

There was no significant change mean diameter and uniformity of the emulsion after about two weeks.

It is to be appreciated that the Detailed Description section, and not the Summary and Abstract sections, is intended to be used to interpret the claims. The Summary and Abstract sections can set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

What is claimed is:

1. An emulsion comprising an emulsifier, an isotonic agent and an oil comprising a docosahexaenoic acid free fatty acid (DHA-FFA) wherein the emulsion is substantially free of eicosapentaenoic acid (EPA) and is suitable for parenteral administration.

2. The emulsion as recited in claim 1, wherein the concentration of the DHA-FFA is about 0.3 mg/ml to about 300 mg/ml of the emulsion.

3. The emulsion as recited in claim 2, wherein the concentration of the DHA-FFA is about 3.0 mg/ml to about 300 mg/ml of the emulsion.

4. The emulsion as recited in claim 3, where the concentration of the DHA-FFA is about 30 mg/ml to 300 mg/ml of the emulsion.

5. The emulsion as recited in claim 2, wherein the concentration of the DHA-FFA is about 20 mg/ml to about 300 mg/ml of the emulsion.

6. The emulsion as recited in claim 2, wherein the concentration of the DHA-FFA is about 140 mg/ml to about 300 mg/ml of the emulsion.

7. The emulsion as recited in claim 3, wherein the concentration of the DHA-FFA is about 140 mg/ml to about 150 mg/ml of the emulsion.

8. The emulsion as recited in claim 1, wherein the mean particle size of the emulsion is less than about 500 nanometers.

9. The emulsion as recited in claim 1, wherein the emulsion comprises about 0.6% to about 10% by weight of the emulsifier.

10. The emulsion as recited in claim 6, wherein the emulsion comprises about 1% to about 4% by weight of the emulsifier.

11. The emulsion as recited in claim 1, wherein the emulsion comprises about 1% to about 5% by weight of the isotonic agent.

12. The emulsion as recited in claim 8, wherein the emulsion comprises about 2.25% to about 2.5% by weight of the isotonic agent.

13. The emulsion as recited in claim 1, wherein the emulsion is substantially free of arachidonic acid (ARA).

14. The emulsion as recited in claim 1, wherein the emulsion comprises about 2% to about 30% by weight oil.

15. The emulsion as recited in claim 11, wherein the oil comprises about 84% to about 95% DHA-FFA of the total weight of the oil.

16. The emulsion as recited in claim 12, where the oil comprises about 95% DHA-FFA of the total weight of the oil.

17. The emulsion as recited in claim 1, wherein the isotonic agent comprises glycerin.

18. The emulsion as recited in claim 1, where the emulsifier is selected from the group consisting of lecithins.

19. An emulsion comprising about 135 milligrams of DHA-FFA per milliliter of the emulsion; about 18 milligrams of a lecithin per milliliter of the emulsion; and about 25 milligrams of glycerin per milliliter of the emulsion wherein the emulsion has a mean particle size of less than about 500 nanometers and wherein the emulsion is substantially free of EPA and is suitable for parenteral administration.

20. An emulsion comprising about 270 milligrams of DHA-FFA per milliliter of the emulsion; about 36 milligrams of a lecithin per milliliter of the emulsion; and about 50 milligrams of glycerin per milliliter of the emulsion wherein the emulsion has a mean particle size of less than about 500 nanometers and wherein the emulsion is substantially free of EPA and is suitable for parenteral administration.
